(19) United States
(12) Patent Application Publication
(10) Pub. No.: US 2016/0113939 A1
(43) Pub. Date: Apr. 28, 2016

(54) Treating Patients with Compounds or Biological Agents That Pass the Safety Tests

(71) Applicant: Jianyi Zhang, Clarksville, MD (US)

(72) Inventor: Jianyi Zhang, Clarksville, MD (US)

(21) Appl. No.: 14/986,622

(22) Filed: Jan. 1, 2016

Related U.S. Application Data
(63) Continuation-in-part of application No. 62/283,743, filed on Sep. 11, 2015.

(51) Int. Cl. A61K 31/55; A61K 31/192; A61K 31/444; A61K 31/185

(52) U.S. Cl. CPC A61K 31/55; A61K 31/185; A61K 31/192; A61K 31/444

(57) ABSTRACT

The invention discloses a method of treatment for various severe disorders which is not treated well by current FDA approved drugs. The therapeutic agents by the method are compounds or biological agents which pass safety tests in the clinical trials, but not pass the efficacy test, as safety of those compounds or biological agents are comparable with any ones in the market which pass both safety and efficacy tests in the clinical trials, combined application of those compounds or biological agents can significantly improve varied severe medical conditions.

### CROSS-REFERENCE TO RELATED APPLICATIONS

The present application relates and claims priority to U.S. provisional patent application No. 62/283,743, filed on Sep. 21, 2015.

### STATEMENT REGARDING FEDERALLY SPONSORED RESEARCH OR DEVELOPMENT

Not Applicable.

### REFERENCE TO SEQUENCE LISTING, A TABLE, OR A COMPUTER PROGRAM LISTING COMPACT DISC APPENDIX

Not Applicable.

### FIELD OF THE INVENTION

The invention relates to treatment of many medical conditions which have no effective treatments or poor results with currently safety-proved, but not efficacy-proved compounds or biological agents.

### ABBREVIATIONS

The term “FDA” refers to the Food and Drug Administration, a USA governmental agency which regulates food and drug in USA.

The term “NCE” refers to New Chemical Entity which is a drug that contains no active moiety that has been approved by the FDA in any other application submitted under section 505(b) of the Federal Food, Drug, and Cosmetic Act.

The term “PTD” refers to Potential Therapeutic Drug, which is compound that passes the safety test, but fails the efficacy test.

SPEAD refers to Safety Passed & Efficacy Approved Drug, which is a compound with approval by FDA to enter the market as a drug to treat medical conditions.

### DESCRIPTION OF THE RELATED ART

Approval of drug products in the U.S. is governed by a rigorous review process conducted by the Food and Drug Administration (FDA). A sponsor must demonstrate a compound or biological agent both safety and efficacy in well controlled clinical trials in the proper patient population for which it is indicated.

Approval of drug products in the U.S. is governed by a rigorous review process conducted by the Food and Drug Administration (FDA). Development of a new drug, especially a new chemical entity (NCE) is a very costly and time-consuming process; it takes roughly 12-15 years and 1.5 billion dollars on average. The high cost of the process makes the development of a new drug with multiple NCEs in same trial almost impossible.

Theoretically there are diseases that can only be effectively treated with different NCEs together; however, the proper combination to treat diseases cannot be done due to high cost the trials make. Conceptually, according to drug treatment, diseases may be roughly divided into three types:

1. Diseases that can be Treated Effectively with a Single Drug

Some diseases can be cured with a single drug. For example, Escherichia coli causes urinary tract infections, Staphylococcus aureus can cause lobar pneumonia, and both of them could be treated successfully with single antibiotics prior to the development of drug-resistant strains.

2. Diseases that can be Treated Effectively by Different Drugs Separately and more Effectively by a Combination of Different Drugs

Different drugs may have a synergistic effect. Hypertension, diabetes, AIDS, asthma, and gastric ulcers belong to this category. There are eight categories of medications to treat hypertension; taking several drugs together can produce a stronger antihypertensive effect. Each medication in the cocktail recipes for AIDS treatment as well as gastric ulcers is an FDA-approved one on the market.

3. Diseases that can Only be Effectively Treated with Different New Compound Entities (NCEs) Together

An NCE approval to enter the market as a drug is called a Safety Passed & Efficacy Approved Drug (SPEAD). We know many NCEs that passed the safety test, but did not show efficacy in clinical trials. This type of NCE might be essential or has the potential to play an important role in treatment of certain medical conditions only if they are included in treatment with other potential beneficial compounds in the trial, which could be called Potential Therapeutic Drug (PTD). PTDs cannot pass the current FDA approved process into the market.

It is impossible to discern what disease falls into this category. We know that there are many severe, terminal conditions such as terminal-stage cancers, autoimmune diseases, and developmental disorders that currently have no effective treatments. Some of them might be well controlled or treated with two or more PTDs, or together with other drugs on the market, and they belong to the third type of diseases.

In the phase II trials, overall success rates were 28% (2006-2007), and 18% (2008-2009). Out of one reported failure, 51% (44 out of 87) were due to insufficient efficacy (Arrowsmith 2011). For every successful phase II trial, there are two compounds that failed efficacy tests.

There were 83 phase III trials and submission failures between 2007 and 2010; failure rate is around 50%. Among those that failed, 66% are due to efficacy (Arrowsmith 2011). The failed NCEs might be used as PTDs for clinical applications.

Semagacestat, tramiprosate, tarenflurbil, and latrepirdine were high-profile, “would-be” Alzheimer's drugs that failed the 3rd phase of clinical trials for efficacy reasons. Each made it through the safety stages (Rafii and Aisen 2009). They might be utilized in clinic as PTDs.

Phylogenetic reconstruction of primary renal carcinomas and associated metastatic sites revealed that 63 to 69% of all somatic mutations not detectable across every tumor region. Intratumor heterogeneity was observed for a mutation within an autoinhibitory domain of the mammalian target of rapamycin (mTOR) kinase (Gerlinger, Rowan et al. 2012).

It is unrealistic to expect one NCE to have desired effects on cancers that have enormous heterogeneity. However, the diseases might be better treated with several NCEs with or without combination with other drugs on the market.

Any compound in the clinical trial is selected from thousand and thousand compounds with specific target. NCEs in the trials usually have different targets. Alzheimer's disease is a devastating neurological disorder that affects more than 37 million people worldwide. Synaptotoxic β-amyloid (Aβ) peptide, the plaques composed of aggregated Aβ, as well as the neurofibrillary tangles composed of hyperphosphorylated tau protein, are held to be essential to the pathogenesis of Alzheimer's disease (AD).

β-amyloid is a peptide with 39 to 43 amino acids. The isoforms with 40 and 42 amino acids (Aβ40/42) are the main constituents of amyloid plaques in the brains of Alzheimer's disease patients. β-amyloid is formed by proteolysis of APP.

Therapeutic strategies include blocking Aβ formation, slowing its aggregation into plaques, lowering its soluble levels in the brain, and disassembling pre-existing amyloid plaques. Semagacestat is a γ-secretase inhibitor; which (along with β-secretase) is responsible for APP proteolysis (Fleisher, Raman et al. 2008). Tarenflurbil is a γ-secretase modulator (Eriksen, Sagi et al. 2003); tramiprosate directly binds Aβ to prevent Aβ aggregation (Gervais 2004). Even they were tested to be safe, but failed to show lack of efficacy in phase III studies, they might significantly improve medical condition of AD in the combination.

PTDs are compounds tested in trials in which safety is comparable with placebo or other SPEADs. Treating patients with them does not increase their risks compared with other drugs on the market.

### SUMMARY OF THE INVENTION

There are a large number of PTDs and combinations of them are unlimited. PTDs are ineffective in the clinical trial separately, but may be effective when they are co-administered with other PTDs or approved drugs. Outcomes of their use might be one of four possibilities:
1) Combination is not secure and ineffective. The combination should not be used in clinical settings.
2) Combination is not safe, but effective. If so, the treating physician must weigh the risk to benefit ratio.

If the combination causes only nausea, vomiting and limb weakness but prolongs life, physicians might continue it. If patients have arrhythmia with the application, that combination should be disbanded.
3) Combination is safe, but ineffective. That is to say, the combination has no effect on the condition, and it should not be used in the treatment.
4) Combination is safe and effective. This is the ideal outcome.

By the FDA regulation, there is neither PTD in the market, nor data to tell us what combination or in what circumstances is effective. With the PTD application, clinicians will soon discern the proper conditions to use PTDs, as well as what combination should not be used for certain conditions.

The clinical impacts of PTDs can be demonstrated in a few weeks or months, if not in a few days. The application might lead to remission of advanced cancer, improvement of autoimmune symptoms, neurologic recovery in stroke patients, and so on.

The informed consent would be necessary prior to treatment and patients should know rationale of PTDs application, and proper monitoring system should be setup to check efficacy with combination among PTDs or PTD with SPEAD, beside severe side-effects, further clinical trial might be optional or necessary to verify the efficacy for the combination of PTDs after their usage.

The targets of PTD treatment are serious medical conditions. The application of PTD might effectively control and improve the condition, or even save lives. Like with any SPEAD, there is always risk of interaction with other drugs; however, they could be detected with monitoring, controlled and eliminated in the practice. Benefits would heavily outweigh risks for patients that receive PTD.

The same principle should also be applied to biological agents, such as vaccines against HIV infection, cancers, etc.

The amendment to the current laws regarding FDA should be made which should allow clinical application of PTD in practice for certain severe or disabiliting medical conditions

### EMBODIMENT

### Embodiment 1

Compounds A, B, C, D underwent phase III clinical trials individually in treatment of Alzheimer's disease, they were all demonstrated safe, but did not show efficacy for improvement of patients' cognitive functions.

These patients were prescribed these compounds with informed consent by different physicians, some of them take A and B, some A and C, some C and D. After 3 months many family members from the patients treated with C, D compounds reported that cognitive functions of their relatives in those medications significantly improved.

Later on, a random clinical trial was done in which C and D combination verse a placebo were included in the study. The result showed cognitive functions in the two groups were significantly different, the patients treated with some C and D compounds were much better than ones in the placebo group (P<0.01) in their cognitive functions, whereas there were no significant differences of side-effects among these two groups.

### Embodiment 2

Compounds X, Y, Z underwent phase III clinical trials individually in treatment of prostate cancer, they were all demonstrated safe, but did not show effective to reduce the tumor size. These patients were prescribed these compounds with informed consent by different physicians, some of them take X plus medication alpha in the market (the first group), some take Y plus medication alpha (the 2nd group), and some take Z plus medication alpha (the 3rd group). Information were collected in central office. Data analysis shows tumor size in the 1st group patients shrinked tremendously, whereas tumor size in other two groups did not have significant change.

A random clinical trial was done in which the first group are compared with these other two groups. The result showed that tumor in the first group was almost un-detectible, whereas the sizes in other groups placebo group had no apparent changes.

### Embodiment 3

Vaccines M, N underwent phase III clinical trials individually for prevention of HIV infection, they were all demonstrated safe, but did not show efficacy to reduce HIV infection.

Vaccines M, N were given to all self-claimed homosexual men in area A, incidence of HIV infection was found to decrease 80% from the previous year among these patients compared with ones who did not receive these vaccines in the area.

A random clinical trial was done in which the first group of patients receive vaccines M, N; the 2nd group of patient received only vaccines M, the 3rd on received placebo. Each group has 1,000 persons.

After one year, it is found that nobody in the first group with HIV infection; 2.1% persons with HIV+ in the 2nd group, and 2.5% persons HIV+ in the placebo group.

### References

Arrowsmith, J. (2011). “Trial watch: Phase II failures: 2008-2010.” Nat Rev Drug Discov 10(5): 328-329.

Arrowsmith, J. (2011). “Trial watch: phase III and submission failures: 2007-2010.” Nat Rev Drug Discov 10(2): 87.

Eriksen, J. L., S. A. Sagi, et al. (2003). “NSAIDs and enantiomers of flurbiprofen target gamma-secretase and lower Abeta 42 in vivo.” J Clin Invest 112(3): 440-449.

Fleisher, A. S., R. Raman, et al. (2008). “Phase 2 safety trial targeting amyloid beta production with a gamma-secretase inhibitor in Alzheimer disease.” Arch Neurol 65(8): 1031-1038.

Gerlinger, M., A. J. Rowan, et al. (2012). “Intratumor heterogeneity and branched evolution revealed by multiregion sequencing.” N Engl J Med 366(10): 883-892.

Gervais, F. (2004). “Gag mimetics: potential to modify underlying disease process in AD.” Neurobiol. Aging 25: S11-12.

Rafii, M. S. and P. S. Aisen (2009). “Recent developments in Alzheimer's disease therapeutics.” BMC Med 7: 7.

1. A method of treatment of medical conditions in human respiratory system by using safety approved, but not efficacy approved compounds or biological agents in the clinical trials.

2. The method of claim 1, wherein said medical conditions are in human neurological system.

3. The method of claim 1, wherein said medical conditions are in human musculoskeletal system.

4. The method of claim 1, wherein said medical conditions are in human digestive system.

5. The method of claim 1, wherein said medical conditions are in human cardiovascular system.

6. The method of claim 1, wherein said medical conditions are in human urinogenital system.

7. The method of claim 1, wherein said medical conditions are in human endocrinological. system.

8. The method of claim 1, wherein said medical conditions are in human blood system.

9. The method of claim 1, wherein said medical conditions are in human immunological system.
